(19) United States
(12) Patent Application Publication
(10) Pub. No.: US 2012/0171338 A1
(43) Pub. Date: Jul. 5, 2012

(54) MEASUREMENT OF A QUALITY OF GRANULAR PRODUCT IN CONTINUOUS FLOW

(75) Inventor: Gabriel Hamid, Loughton (GB)

(73) Assignee: Buhler Sortex Ltd., London (GB)

(21) Appl. No.: 13/318,041

(22) PCT Filed: Dec. 16, 2009
(86) PCT No.: PCT/GB2009/002897

§ 371 Date: Mar. 7, 2012

(30) Foreign Application Priority Data
Apr. 30, 2009 (GB) 0907526.8

(51) Int. Cl. G01N 21/85; G01N 21/55; G01J 3/50; G01N 33/02

(52) U.S. Cl. 426/231; 356/445; 356/402

(57) ABSTRACT

In apparatus for measuring the quality of a granular product in continuous flow, the product is allowed to move along a channel having a window set in the channel boundary contacted by the moving product. An optical system monitors product in the channel. A light source illuminates product in the channel through the window, and a sensor receives light reflected from the product through the window in at least two wavelength ranges. A processor receives signals from the sensor representative of the quantity of reflected light received in the respective wavelengths, and compares the respective signals to generate a measurement of the product quality. The window will normally be disposed in the underside of the channel, with the channel set at an angle of at least 45° to the horizontal to allow product to move there along under gravity, and against the window.

This invention relates to the measurement of a quality of granular product in processing apparatus in which the product is being treated or sorted, or merely subject to inspection. It has particular application to rice, which is commonly subject to milling to establish and enhance a level of whiteness in the final product.

Rice is a staple food for over half the world's population. Despite the nutritional benefits of brown rice compared to white rice, the majority of people prefer to eat white rice. White rice is made by removing the outer layers of bran from the rice kernel. This is normally accomplished by milling, and it is the millers' art to remove sufficient bran whilst minimising the loss of good rice through either breakage or over-milling.

There are significant benefits to be gained from optimising the performance of the milling process. These benefits are economic in terms of higher yields; environmental in terms of reducing power consumption and wastage; and health in terms of gentler milling to retain some of the nutrients and vitamins in the outer layers of the kernel. However, as a rice mill has many stages of milling machinery, with many degrees of freedom, and the properties of the input rice to the mill are subject to considerable variation, optimising the performance of the mill, whilst maintaining the quality of the milled rice is a complex task.

In order to control a multi-stage milling process, it is necessary to know either the Degree of Milling or the whiteness at each stage. There are well-established standards within the Rice Industry for measuring the Degree of Milling and the whiteness of rice. The Degree of Milling is a quantification of the amount of bran remaining on the rice and is measured by chemical analysis of the rice. The whiteness of rice covers two physical properties of rice. The first is the colour (or hue) of the rice. In general terms, as the rice is milled the colour changes from brown to yellow to white. The second is gloss. Once the bran is removed, the whiteness of the rice can be further increased by polishing the rice to make it more glossy, i.e. give it a higher reflective index.

There are many well-known scientific systems for measuring whiteness, such as CIE Whiteness, the Hunterlab Whiteness Index or Ganz Whiteness. A commonly used technique is based on the reflection of blue light. The Degree of Milling and whiteness of rice are loosely correlated. The present invention is directed at the measurement of the reflectivity of a product as a guide to product quality.

Various devices are known for measuring the reflectivity of a product and particularly granular products. There are static devices adapted to receive a discrete sample which is then illuminated and its reflectivity measured directly. Apparatus has also been proposed in which reflectivity of granular product in a flow path is measured, and in this respect reference is directed to U.S. Pat. Nos. 4,483,244, and 5,406,084, the contents of which are hereby incorporated by reference. The first of these patents; U.S. Pat. No. 4,483,244 is directed specifically at apparatus for whitening rice, and in which the passage of rice is halted at intervals for a reflectivity measurement to be taken. U.S. Pat. No. 5,406,084 discloses a technique for measuring a variety of characteristics of food products using reflective techniques.

The present invention is directed at apparatus for measuring a quality of a granular product in equipment in which the product is moving. Specifically, the invention is directed at apparatus which enables measurement of the respective product quality while the product is in continuous flow. The apparatus has a channel for the passage of the product. The product may be driven or drawn along the channel, but the channel is normally set at a sufficient angle to the horizontal to allow product to move along it under gravity. The channel can be the discharge duct from a product hopper onto a chute feeder, or the chute itself. A window is set in the channel boundary for contact with product moving in the channel, and an optical system is disposed adjacent the window for monitoring product descending the channel through the window. The optical system comprises a light source for illuminating product in the channel through the window; a sensor adapted to receive light reflected from the product through the window in at least two wavelength ranges; and a processor coupled to the sensor to receive signals therefrom representative of the quantity of reflected light received in the respective wavelengths, the processor being programmed to compare the respective signals from the sensor to generate a measurement of said quality of the product.

The invention is also directed at a method of measuring such a granular product quality as is referred to above, and using the apparatus just described. As the product flows along the channel and across the window, it will engage the window and this engagement can serve to keep the window clean and ensure good transmission of illuminating and reflected light through the window. In this context it is noted that food products particularly moving at high speeds along chutes or channels are very effective in themselves keeping the respective chute or channel surfaces clean. One aspect of the cleaning process is the continuous movement of product over the respective surface. For this reason, installation of the window in the channel of apparatus of the invention should be conducted with great care, to ensure that there is a smooth transition between the chute surface and the material, normally glass, of the window. In some circumstances it can be appropriate to increase the flow of product occasionally, or just prior to generating a quality measurement, to maximise the cleaning effect.

In the practice of the invention signals from the sensors are representative of the quantity of reflected light received in each of at least two wavelength ranges; typically selected from the green, blue and red wavelength ranges. The signals can be compared in various ways. As described hereinafter, a measurement of the ratio of the signals representative of the quantities of reflected light in the blue and green wavelength ranges provides an indication of relative whiteness. However, other comparisons and ratios can be used. The ratio of the signal representing reflected light in one wavelength range to the sum of the signals representing reflected light in two or more other wavelength ranges can be a useful indicator of a product quality. For example a reflected blue light signal can be compared to the sum of the reflected green and red light signals or to the sum of the reflected signals in all three primary colours.

The window in the channel in apparatus of the invention is typically disposed on the underside of the channel. This means that a proportion of the weight of the flowing product is applied to the window surface to maximise the cleaning effect. However, it can be disposed in a side wall of the channel, directly opposite a facing side wall, or in an inclined channel face. What is important in this respect is that there is a sufficient quantity of product against the window to ensure that the reflected light comes from the product and not from a surface behind the product. To ensure this, it can be desirable to maintain a minimum depth of the stream of product in the channel, and/or to ensure that any background surface is entirely neutral and non-reflective. If the window is disposed in the side of a channel opposite a facing side wall, then of course the minimum depth must be the height of the window. The disadvantage of this arrangement is of course that there is less pressure between the product in the channel and the window surface. To meet this problem, or to ensure in any event that there is sufficient product against the window, one or more baffles may be included for directing product descending the channel towards the window.

In order to preserve the cleanliness of the product subject to quality measurement, the channel in apparatus according to the invention will normally be closed, and have a circular or elliptical cross-section to avoid sharp angles in which product might be held. However, a square or rectangular cross-section can be acceptable if the channel is at a sufficient incline to the horizontal. A portion of the cross-section will normally be flat to receive the window. While curved windows might be used, such windows can distort the passage of light thereby rendering unnecessarily complex the processing required to obtain a quality measurement.

The optical system will normally be a closed unit mounted on the external surface of the channel, and substantially sealed against the ingress of air or foreign matter. In this way, the illuminating and reflected light are not compromised by dust or other airborne pollutants. The light source will normally be a source of white light, but the system may include one or more filters to restrict the light transmitted to the window to the selected wavelength ranges. If white light is to be used to illuminate the product behind the window, the illuminating or reflected light must be divided into spectral components to generate signals for use in the practice of the invention. White light or light in two distinct wavelength ranges may be directed at products behind the window, and the sensors adapted to monitor the reflected light in two correspondingly distinct wavelength ranges. In a particular embodiment, light in the two wavelength ranges can be directed at the window alternately from two separate elements, such as flashing LEDs, with the reflected light being monitored by a single sensor. However they are generated, the two signals generated by the sensor or sensors are then compared in the processor and a ratio of the two signals used as the basis for the product quality measurement. The advantage of using this ratio technique is that it is less sensitive to variations in the depth of product over the window, to extraneous light variations such as reflections from within the channel, or to temporary variation of the transmissivity of the window as a consequence of dust etc. engaging the window in the channel. An advantage of being less sensitive to variation in product depth over the window is that a fixed depth as is provided by a choke feed, is not required. When the ratio technique is used, the preferred wavelength ranges for the reflected light are those for blue and green. However, other colours might be used.

As noted above, the channel in apparatus according to the invention is normally inclined to the horizontal, typically at an angle of at least 45°. While it is preferred to ensure that the granular product is not in freefall down the channel, such movement can of course be controlled by the use of baffles as described above. Thus, in some applications of the invention a vertical channel could be used, but normally in a defined section of the pipe in which the rate of flow would be slowed, but not halted.

The invention may be exploited in product processing systems either as a separate element of such systems, or as an adjunct thereto. If a system already includes a flow path that can be adapted to form the channel in apparatus according to the invention, then the apparatus can be installed in the existing system. Alternatively, such a system can be adapted to create a secondary flow path for diverting product from a main flow path, and the secondary flow path adapted to form the channel in apparatus according to the invention.

When practising the invention, product quality will normally be repeatedly measured at intervals while the product is flowing, and an average measurement calculated for a given period of product flow. In the multi-stage processing systems, apparatus of the invention can be installed at different stages to provide a comprehensive analysis of product quality at different stages in the process. This is particularly valuable in the processing of rice as it is progressively treated initially to remove bran and subsequently by polishing.

The invention will now be further described by way of example, and with reference to the accompanying schematic drawings, wherein:

FIG. 1 is a longitudinal cross-section through the channel in apparatus according to the invention;

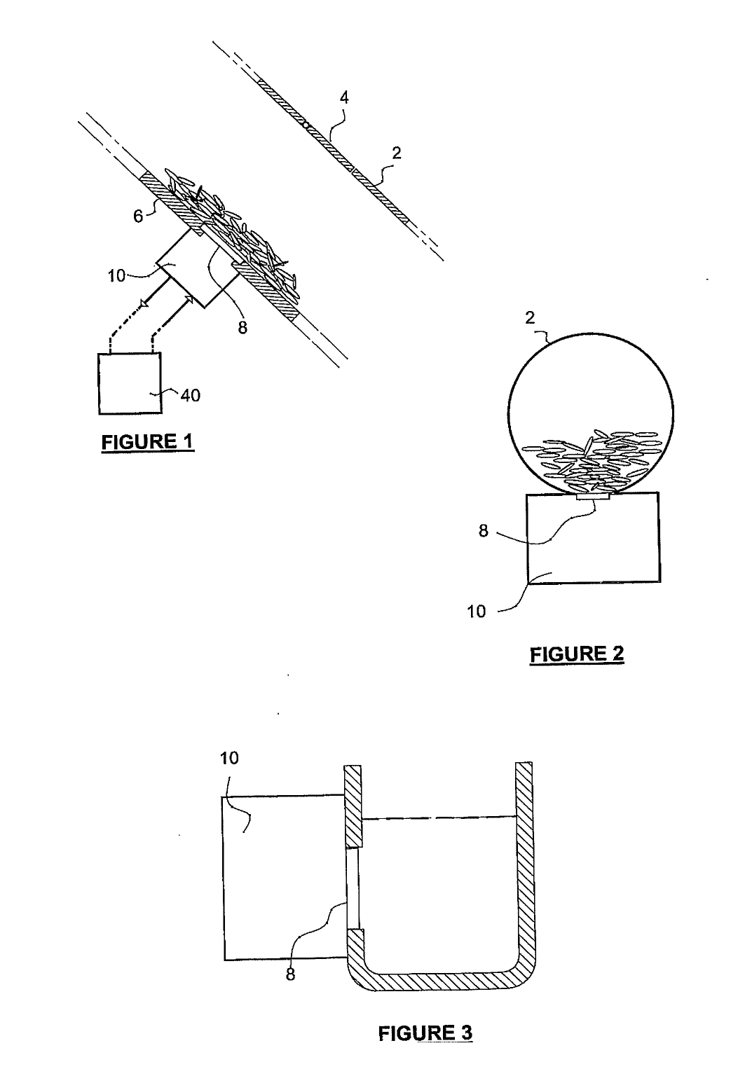

FIG. 2 is a lateral cross-section through a channel of the kind shown in FIG. 1;

FIG. 3 is a lateral cross-section through an alternative channel suitable for exploitation of the invention;

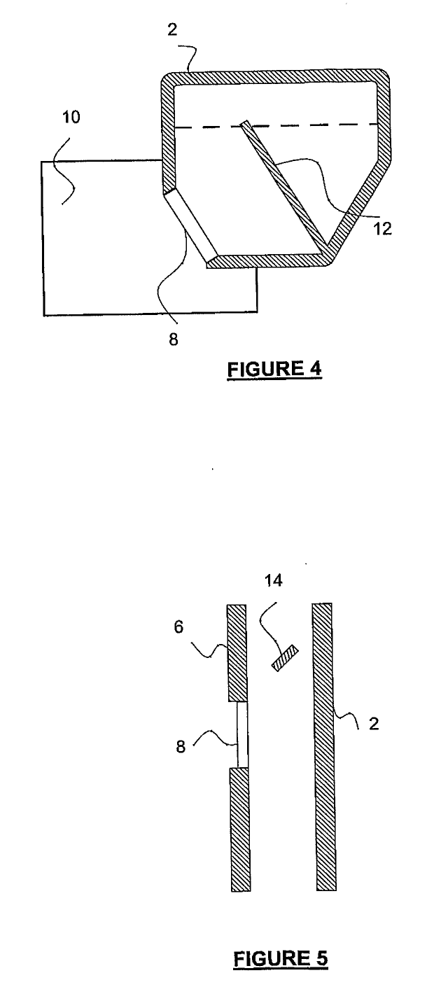

FIG. 4 is a lateral cross-section through another alternative channel cross-section;

FIG. 5 illustrates how a baffle may be used to direct product towards the window in a vertical channel wall;

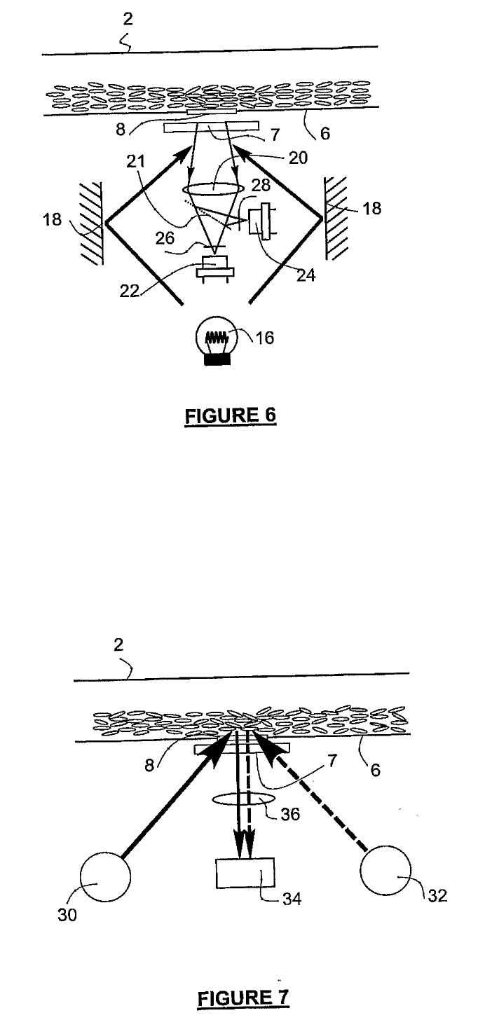

FIG. 6 illustrates an optical system for illuminating the window with light from a single source;

FIG. 7 illustrates an optical system in which the window is illuminated with light from separate sources;

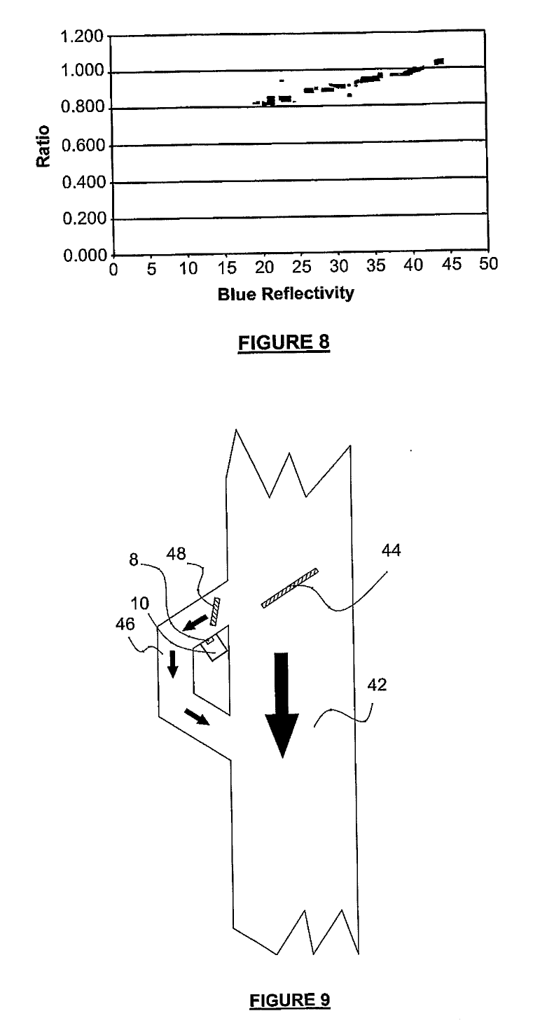

FIG. 8 is a graph in which the ratio of reflected blue light to reflected green light is plotted against whiteness; and

FIG. 9 illustrates how apparatus of the invention can be installed in a product processing system.

The apparatus according to the invention described with reference to the accompanying drawings is primarily for use in measuring the whiteness of rice in continuous flow, but it will be appreciated that it will also be suitable for other flowable granular products. FIG. 1 illustrates a typical channel in apparatus of the invention having an upper wall 2 fitted with an access door 4, and a lower wall 6 fitted with a window 8. The material of the channel is preferably stainless steel or hard anodized aluminium, and the preferred window is of glass. In order to avoid disrupting the flow of rice over the window, it should insofar as is possible, define a surface that is flush with the adjacent internal surface of the channel. Most importantly the upstream edge of the window should not project above the surface of the lower channel wall 6, as this would interfere with the product flow. An optical system indicated at 10 is attached to the lower wall 6 of the channel. The optical system will normally be a substantially sealed unit fixed to the channel wall in such a manner as to prevent the ingress of air or airborne material which could compromise the optical units in the system, and the transmission of light within it. It is of course essential that the respective surfaces within the optical system are kept clean and free from dust and other material, to ensure accuracy of measurement.

The rice flowing in the channel over the window 8 creates a volume of product, such as a mass of rice, over the window, and its light properties can be optically monitored, as will be described in more detail below. The moving rice also performs a continuous wiping effect on the glass window, keeping it clean. It is important in this respect that the rice in the channel maintains continuous movement while optical measurements are being taken. If it were to become stationary, then there is a risk that dust will lodge on the window, compromising the optical signals.

FIG. 2 illustrates the lateral cross-section of the channel, which is shown as circular, and the product flowing in the channel over and on either side of the window 8. A significant depth of rice over the window is required to enable the optical system to take accurate measurements. A preferred minimum depth for rice is six grains arranged randomly over the window. Preferably it will be more, but should in any event be kept substantially constant. Whatever the depth in the channel, in order to minimise the impact of extraneous light being reflected from within the channel, the internal face of the channel opposite the window should ideally be neutral and non-reflective.

FIG. 3 illustrates a square or rectangular channel cross-section, in which the window 8 is installed in one side wall. Provided the depth of product in the channel is above the upper boundary of the window 8, then optical measurements can be accurately taken, and of course the depth of rice providing basis for the optical measurement is the lateral dimension of the channel. While this arrangement has the advantage of ensuring this constant depth of rice, there is less pressure applied by the rice to the window, and the cleaning effect is reduced relative to that in the channel of FIGS. 1 and 2. FIG. 4 shows another alternative cross-section which ensures better engagement of the flowing product with the window, relative to that of FIG. 3, but the depth of rice providing the basis for the optical measurements is not consistent. This can be avoided by installing a baffle 12 to define an internal channel of constant depth over the window 8.

In the extreme case of a vertical channel, one or more baffles 14 as shown in FIG. 5 can be used to good effect. Baffle 14 can direct flowing product towards the wall 6 of the channel, and thereby ensure there is sufficient product flowing over the window for useful measurements to be taken.

As noted above, the optical system in apparatus of the invention is normally in the form of a sealed unit attached to the outer surface of the channel around the window 8. FIG. 6 illustrates one arrangement for illuminating the window and monitoring the light reflected from the flowing product through the window. Light from a single source 16 is reflected by mirrors 18 so as to impinge on the window 8 at an angle of around 45° to illuminate product within the channel on the other side of the window. Light reflected from the product passes back through the window and is collected by a focusing lens 20. The focused light is split at 21 and collected by two separate detectors 22 and 24, through filters 26 and 28. The filters are normally for blue and green light. Thus, detector 22 receives reflective light only in the blue wavelength range, and detector 24 receives reflected light only in the green wavelength range. Each of the detectors generates a signal representative of the quantity of reflected light received, and these signals are transmitted to a processor, indicated at 40 in FIG. 1, but not shown in FIG. 6.

FIG. 7 illustrates an alternative optical system in which two separate light sources 30 and 32 are used. These light sources are of different wavelength ranges and are pulsed on and off alternately by a sequencer (not shown), each directing light onto window 8. The light illuminates product on the other side of the window which in turn reflects light back through the window and is collected by a focussing lens 36. The focused light is collected by a single detector 34. The detector decodes two signals representative of the quantity of reflected light received by synchronisation with the two separate light sources. These two signals are transmitted to a processor, indicated at 40 in FIG. 1 but not shown in FIG. 7.

The processor generates a measurement of the quality of product in the channel as the ratio of the reflected light at the two different wavelength ranges. The processor can be first calibrated by the use of a reference plate 7 initially disposed over the window outside of the channel and out of contact with the flowing product, and if the reference plate is of a standard whiteness say, the whiteness of product in the channel relative to that of the reference plate, can be calculated as follows:

$$Whiteness = \frac{1}{m} \{ \frac{B \cdot {G}_{ref}}{{B}_{ref} \cdot G} - c \}$$

where B represents the light reflected from the product in the blue wavelength range; G represents light reflected from the product in the green wavelength range; B_{ref} and G_{ref} represent the quantity of light reflected in the blue and green wavelength ranges from the reference plate; and m and c are constants.

From the above equation, it will be apparent that the relationship between whiteness and the ratio of the quantities of reflected light in the blue and green wavelength ranges is linear. This linear relationship is illustrated in FIG. 8 in which the ratio was plotted against whiteness or blue reflectivity for different varieties of rice: That, Jasmine and Basmati. When adapted to the above equation, the calibration values of m and c are given by the gradient and intercept of the best fit line to the measurements used. We have though, found that different calibration values must be used if the rice has been cooked or part-cooked, with the consequence that its colour and reflectivity has been significantly altered.

As noted above, apparatus according to the invention will normally be used to measure the respective product quality by taking measurements at intervals while product is flowing in the channel. The measured values can be used for different kinds of analysis, but normally an average will be taken as an overall quality guide. In a product processing system such as a rice mill, having a plurality of processing stages, apparatus according to the invention can be readily installed at each stage, without major alteration being required.

While apparatus of the invention can be installed on a main flow path for product in a processing system, the system can be provided with a secondary flow path for diverting product from the main flow path, with apparatus according to the invention being installed on the secondary path. A simple arrangement for accomplishing this is shown in FIG. 9 in which product flowing in a main path 42 is deflected by baffle 44 into a secondary flow path 46. A second baffle 48 may be used to redirect product in the secondary path such that it flows directly over the window 8 and optical system 10 mounted on the secondary flow path. The product will though, remain in continuous flow over the window and will be recycled to the main flow path 42 thereafter.

1. Apparatus for measuring a quality of a granular product in continuous flow, in which a channel for passage of said product is adapted to allow product to move therealong, and has a window set in the channel boundary for contact with product moving in the channel, the apparatus including an optical system for monitoring product in the channel, which optical system comprises a light source for illuminating product in the channel through the window; a sensor adapted to receive light reflected from the product through the window in at least two wavelength ranges; and a processor coupled to the sensor to receive signals therefrom representative of the quantity of reflected light received in the respective wavelengths, wherein the processor is programmed to calculate a ratio from the respective signals from the sensor to generate a measurement of said quality of the product

2. Apparatus according to claim 1 wherein the channel is set at an angle to the horizontal to allow product to move therealong under gravity.

3. Apparatus according to claim 2 wherein said angle is at least 45°.

4. Apparatus according to claim 1 wherein the window is disposed in the underside of the channel.

5. Apparatus according to claim 1 wherein the window is disposed in a side wall of the channel.

6. Apparatus according to claim 5 wherein the window is disposed directly opposite a facing side wall of the channel

7. Apparatus according to claim 1 wherein the channel is an open channel

8. Apparatus according to claim 1 including a baffle for directing product moving in the channel towards the window.

9. Apparatus according to claim 1 wherein the optical system is a closed unit mounted on the external surface of the channel and substantially sealed against the ingress of air and foreign matter.

10. Apparatus according to claim 1 wherein the light source is a source of white light, and the optical system includes means for directing only light in specific wavelength ranges onto the sensor.

11. Apparatus according to claim 10 wherein the filters are in the path of the reflected light.

12. Apparatus according to claim 1 wherein the light source comprises separate elements for emitting light in different wavelength ranges.

13. Apparatus according to claim 12 wherein the elements are light emitting diodes.

14. Apparatus according to claim 12 including a sequencer for pulsing the elements alternately, the sensor comprising a single detector synchronised with the sequencer to decode the respective signals.

15. Apparatus according to claim 1 wherein one of said wavelength ranges of reflected light is that for blue light.

16. Apparatus according to claim 1 wherein one of said wavelength ranges of reflected light is that for green light.

17. A method of measuring a quality of a granular product in continuous flow comprising passing the product along a channel having a window in its wall against which product engages as it moves therealong; illuminating product in the channel through the window; detecting light reflected through the window separately in at least two wavelength ranges to generate signals representative of the respective quantities of reflected light received in each wavelength range; and transmitting the signals to a processor wherein the processor calculates a ratio from the generated signals as a measurement of said quality of the granular product.

18. A method according to claim 17 wherein the reflected light is detected in two wavelength ranges, and the processor calculates the ratio between the signals generated therefrom.

19. A method according to claim 17 wherein the channel is set at an angle to the horizontal and product moves therealong under gravity.

20. A method according to claim 17 including the step of maintaining a minimum depth of product against the window as it passes thereover.

21. A method according to claim 20 wherein the minimum depth is maintained by a baffle directing product towards the window as it moves along the channel.

22. A method according to claim 17 wherein the flow of product is diverted from a main stream of product for quality measurement.

23. A method according to claim 17 wherein one of said wavelength ranges is that of blue light and the other is that of green light.

24. A method according to claim 17 wherein the product quality is repeatedly measured at intervals while the product is flowing, and an average measurement is calculated for a given quantity of product.

25. A method according to claim 17 wherein the granular product is rice, and the product quality being measured is whiteness.

26. Processing apparatus for a granular product in which the product is in continuous flow in a section of the apparatus, which apparatus includes a main flow path for said product and apparatus according to claim 1 wherein the channel forms part of the main flow path.

27. Processing apparatus for a granular product in which the product is in continuous flow in a section of the apparatus, which apparatus includes a main flow path for said product, and a secondary flow path for diverting product from the main flow path for quality measurement; and apparatus according to claim 1 wherein the channel forms part of the secondary flow path.

28. Processing apparatus according to claim 26 having a plurality of processing stages, in which apparatus according to claim 1 is installed in each processing stage.
